(19) United States
(12) Patent Application Publication
(10) Pub. No.: US 2025/0271978 A1
(43) Pub. Date: Aug. 28, 2025

(54) Controlling Multiple Views of an Image Data Set and User Interfaces for AR Headsets

(71) Applicant: Novarad Corporation, Provo, UT (US)

(72) Inventors: Wendell Arlen Gibby, Mapleton, UT (US); Steven Todd Cvetko, Draper, UT (US)

(21) Appl. No.: 19/006,172

(22) Filed: Dec. 30, 2024

Related U.S. Application Data
(60) Provisional application No. 63/559,158, filed on Feb. 28, 2024.

(51) Int. Cl. G06F 3/04815; G06F 3/01; G06F 3/0484

(52) U.S. Cl. CPC G06F 3/04815; G06F 3/011; G06F 3/0484

(57) ABSTRACT

Technology is described for a method and system for managing a first user view and a second user view of an image data set aligned with a body of a person using AR headsets. The method can include the operation of determining the first user view of the image data set based in part on a position of a first user in a 3D coordinate space defined using a first AR headset. A second user with a second AR headset in the 3D coordinate space may be identified. The first user's position in the 3D coordinate space with respect to the person may be sent to the second AR headset. The second user view may be set to the first user view of the image data set through the second AR headset.

### PRIORITY DATA

This application is a continuation of U.S. Provisional Patent Application Ser. No. 63/559,158, filed on Feb. 28, 2024, which is incorporated herein by reference.

### BACKGROUND

Mixed or augmented reality is an area of computing technology where views from the physical world and images from virtual computing worlds may be combined into a mixed reality world. In mixed reality, people, places, and objects from the physical world and virtual worlds become a blended visual and audio environment. A mixed reality experience may be provided through existing commercial or custom software along with the use of VR (virtual reality) or AR (augmented reality) headsets.

Augmented reality (AR) is an example of mixed reality where a live direct view (or an indirect view) of a physical, real-world environment is augmented or supplemented by computer-generated sensory input such as sound, video, graphics or other data. Augmentation is performed as a real-world location is viewed and in context with environmental elements. With the help of AR technology (e.g. adding computer vision and object recognition) the information about the surrounding real world of the user may become interactive and may be digitally modified.

### BRIEF DESCRIPTION OF THE DRAWINGS

FIG. 1 is a schematic diagram illustrating an example of a system and method for managing a first user view and a second user view of an image data set, where a view of the image data set is aligned to a person's body.

FIG. 2 is a schematic diagram illustrating an example of a system and method for managing a first user view and a second user view of an image data set, where a view of the image data set is positioned at a location determined by a user of an AR headset.

FIG. 3 illustrates an example of a slice of a 3D image data set that may be displayed as an overlay to anatomy.

FIG. 4 illustrates an example of a slice of a 3D image data set that may be displayed as an overlay to anatomy and an example navigational view using the AR headset.

FIG. 5 is a flowchart illustrating an example method for managing a first user view and a second user view of an image data set.

FIG. 6 is a block diagram that provides an example illustration of a computing device that may be employed in the present technology.

### DETAILED DESCRIPTION

Reference will now be made to the examples illustrated in the drawings, and specific language will be used herein to describe the same. It will nevertheless be understood that no limitation of the scope of the technology is thereby intended. Alterations and further modifications of the features illustrated herein, and additional applications of the examples as illustrated, which would occur to one skilled in the relevant art and having possession of this disclosure, are to be considered within the scope of the description.

A technology is described that may provide a shared interface for users (e.g., medical professionals or doctors) who are using AR headset technology in medical settings. Specifically, this technology may allow medical professionals to use AR headsets to see the user interface and portions or all of image data set(s) aligned with a patient during a medical procedure as seen by a selected doctor through a different AR headset. This selected doctor or medical professional may be the designated medical professional for performing a medical procedure. This way more than one medical professional can share the experience that a selected or designated medical professional is having through the AR headset.

Each doctor in the same 3D coordinate space may see the same view from their own perspective. For example, the doctor may slice the image data set (e.g., a CT scan or MRI scan) to create a sagittal view. Then the other doctors in the 3D coordinate space can see the same view (i.e., the same data cut) from the perspectives where they are located. This is useful because the image data may be aligned to the body of a person or patient. Alternatively, the other doctors can see the 3D slice of the image data set from the perspective of the designated doctor, and this view may be at the location where the slice was originally created with a new orientation toward the viewing doctor (of course the slice may not remain aligned with the patient's body). The slice may also be viewed at another location in the 3D coordinate space that is different than where the 3D slice was originally created. The slice may be moved (or translated) to a new coordinate location and may be rotated so that the viewing doctor sees the same view of the slice as the designated doctor.

The AR hardware and processes may track where each AR headset is in the 3D coordinate system and may track a headset pose (e.g., direction the AR headset is looking) in the 3D coordinate system. A second AR headset can be used to control the view and navigation for a first AR headset as though the first AR headset is being actually worn by the person wearing the second AR headset and the second AR headset is at the same location as the first AR headset. Because the user of the second AR headset may view the data as though the second AR headset is at the same perspective and position as the first AR headset, then the second user or medical professional may then adjust the spatial positioning, brightness, transparency, slice location, etc. for portions or all of the image data set. These adjustments may also be propagated back to the first AR headset and any other additional AR headsets in the 3D coordinate system that are viewing the image data set. This may mean that a second AR headset can control the other person's view of the image data set through a first AR headset. The first AR headset may also share control of the navigation of the image data set and how the image data set is reconstructed for a user or medical professional to view (e.g., a 3D view, slice view, view orientation, etc.)

In addition, each user or medical professional may be presented with multiple smaller navigation view panels or break-out views that may be inset in the AR headset's view to provide a multi-navigation screen. The navigation views may display a view of the image data set from a perspective that is different than the user's actual view point (e.g., a side view, a mirror image view, a top view, bottom view, etc.).

This technology can transfer the perspective of virtual objects in the AR headsets between separate AR headsets without having to transfer the data contained in a projection of the 3D image data set or without sending video of what one AR headset is seeing or doing. Sending large graphical data sets can consume a lot of bandwidth and/or use a high speed wired connection. Most operating rooms in the US and especially in foreign countries cannot guarantee high speed wired connections to transfer large graphical data sets or video in real time between AR headsets in the same operating room. Accordingly, transferring the positional data of the AR headsets, an instrument position, the patient orientation, a user interface state, etc. to an attending physician's headset is useful in the training and assistance of other medical professionals. Further, the additional AR headsets may have the alignment of an image data set to a body of a person or patient in the operating room transferred to them so the additional AR headsets do not have to go through the registration and alignment process performed by the first AR headset (or at least one other AR headset).

The present technology includes a system and method for managing a first user view and a second user view of an image data set aligned with a body of a person using AR headsets. The image data set may be aligned with anatomical structures of the person using at least one marker (e.g., an optical marker) on the person, using morphometrics, using radiopaque markers, or other alignment methods referenced in this disclosure. An image data set can be aligned to the body of the person using a marker or other alignment systems. Medical imaging may be obtained and aligned with a body of a person. For example, a CT (computed tomography) scan, MRI (magnetic resonance imaging) image or other imaging may be overlaid on the patient and used as a reference for aspects of a patient's anatomical structure being operated on. U.S. Pat. Nos. 9,892,564; 10,475,244; 11,004,271; 10,010,379; 10,945,807; 11,266,480; 10,825,563; 11,237,627; 11,287,874; U.S. patent application Ser. No. 17/706,462 entitled “Using Optical Codes with Augmented Reality Displays”; and U.S. patent application Ser. No. 17/536,009 entitled “Image Data Set Alignment for an AR Headset Using Anatomic Structures and Data Fitting”; and U.S. patent application Ser. No. 17/978,962 entitled “3D Spatial Mapping in a 3D Coordinate System of an AR Headset Using 2D Images” describe methods and systems for aligning an image data set from medical imaging devices with a body of a person and these descriptions are incorporated in their entirety by reference herein. An image data set may be aligned to the body of the person using: markers, optical codes, radiopaque markers, 2D imaging, morphometrics or other systems for alignment of 3D image data sets. These patents also describe a wide variety of medical imaging types that may be used to obtain 3D image data sets.

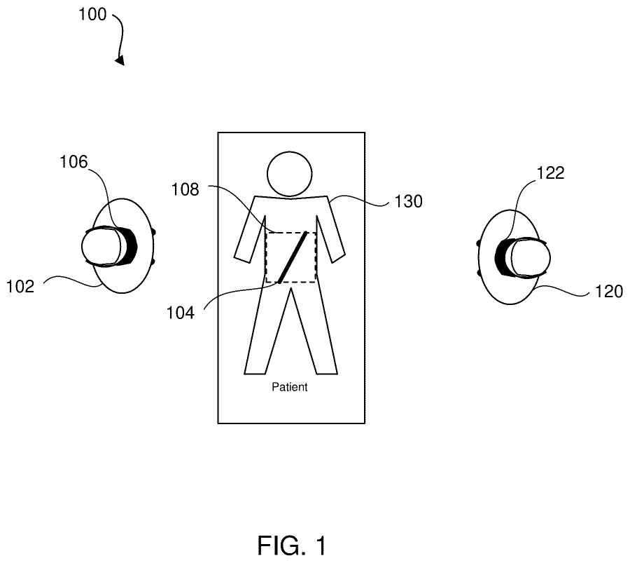

FIG. 1 illustrates that a first user view of the image data set 108 may be determined based in part on a position of a first user 102 in a 3D coordinate space 100 as defined using a first AR headset 106. A second user 120 with a second AR headset 122 having a second user view may be identified in the 3D coordinate space 100. The first user view and the second user view may include information about a pose of the AR headset and a coordinate location of the AR headset (e.g., each AR headset). Accordingly, the first AR headset and the second AR headset may use a common 3D coordinate system in a location. The 3D coordinate system may be set by a single AR headset or through negotiation of the AR headsets. The location of the AR headsets may be in an operating room, examination room, training room, clinic, hospital room or another location where a medical procedure may be desired to be performed on the body of a person.

The first user's position in the 3D coordinate space with respect to the person may be sent to the second AR headset. The first user's position may be a Cartesian coordinate or another coordinate in the 3D coordinate space (e.g., polar coordinates or another coordinate space system) that is detected using the first AR headset 106.

The second user view 124 of the image data set or other virtual objects may be set to the first user view of the image data set or other virtual objects through the second AR headset. This may mean that second user can see the same 3D slice 104 or projection from the CT scan or MRI scan and the 3D slice may be viewed from the same side so the second user will see the slice in the same way that that the first user is viewing the slice, but the 3D slice may not be aligned with the patient's anatomy. The second user may initially be viewing the 3D slice from a different angle or position than the first user but the first user's viewpoint of the image data set and virtual objects can be transferred to the second user's AR headset.

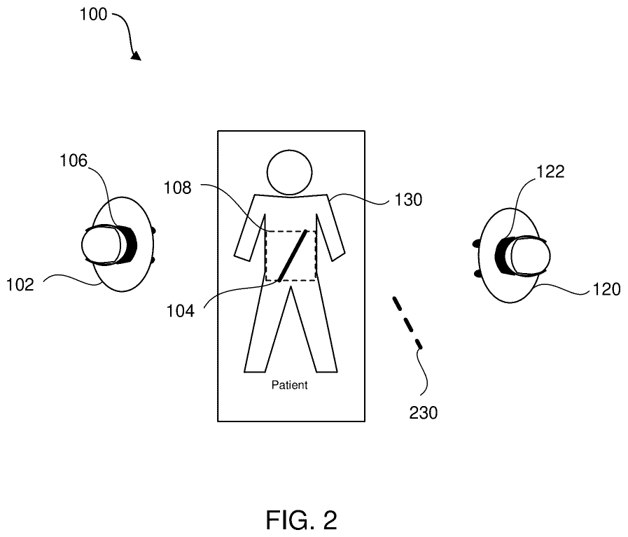

In one example, FIG. 2 illustrates that the 3D slice 230 may be presented to the second user but not overlaid on the patient as viewed through the AR headset. The 3D slice 230 may be located in a position that is selected by the second user and the 3D slice 230 may be viewed with the same slice and orientation from the image data set that the first user is using. The 3D slice 230 may be overlaid on the patient, if desired, but the 3D slice 230 may not be aligned with the anatomical features of the patient 230 in order for the second user to view the 3D slice with the same orientation as the first user. The goal can be for the second user to see the virtual view of image data set or other virtual objects that the first user is seeing in the first user's AR headset. More specifically, at least a portion of the image data set may be displayed from a perspective that matches the first user view of the image data set. In one example, the portion of the image data set may be a slice (e.g., 3D slice or a single layer of voxels) from the image data set.

In another example, a first user may be allowed to control a second user view of the image data set or the first user may control navigation of the second user. This means that when the first user moves the 3D slice through the CT scan or MRI scan then the 3D slice will also move for the second user.

In another example, a second user may be allowed to control a first user view of the image data set. If the second user moves the 3D slice by using hand gestures or finger gestures, the view of the 3D slice may be moved for the first user. Any navigation changes made by the second user can be propagated to the first user's AR headset.

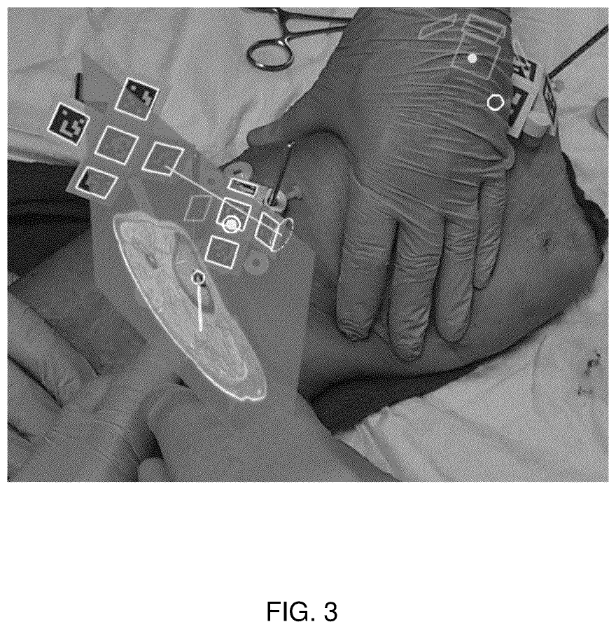

The user interface may also be configured so that can both the first user (e.g., doctor) and the second user (e.g., doctor) can each alter a 3D slice position and orientation, drag the slice through the image data set to see different slice layers, rotate the cut, etc. These shared controls may apply to any number of users or medical professionals in the same 3D coordinate system. The other users or medical professionals who have connected to the primary user's sessions can see the virtual elements in the AR headset from their own perspective or the primary doctor's perspective. FIG. 3 illustrates a slice of the image data set that may be viewed from a first user's perspective, and the use of a medical instrument and representation of a virtual medical instrument (e.g., a burr) on a patient's anatomy (i.e., a cadaver in this image).

Data representing the first user view may also be sent to the second AR headset of the second user, and the data may include one or more viewing details. Examples of these viewing details may include sending information about at least one of: the existence of at least one projection slice viewed from a perspective of the first user, a location and/or orientation of the projection slice, a location of the first user in the 3D coordinate space, a depth of at least one projection slice, a medical device location, object locations, a pointer location, or other virtual object and physical object locations.

In another example of the technology, the first user and the second user may share user interface functions from their own perspectives. A user interface function may include at least one of: altering a position of the image data set in the 3D coordinate system, moving to a different slice in the image data set, rotating a projection slice, changing a projection angle of a slice; identifying a slice of the image data set, and using graphical user interface (GUI) controls from their own perspective. Similarly, the second user may perform functions from a perspective of the first user, including the functions listed above. This means the second user may see what virtual objects the first user is seeing and be able to do operations like altering a position of the image data set from the first user's perspective, dragging or rotating a slice in the image data set from the first user's perspective, or using graphical user interface (GUI) controls from the perspective of the second user.

In the situation where a first user is using a set of graphical user interface (GUI) controls to perform tasks with respect the image data set that is overlaid on the patient, a copy of these same GUI controls may also be presented to the second user, while facing the second user, so that the second user can see the things the first user is doing with the GUI interface. For example, if the first user presses virtual button A, then the second user will see their own copy of the interface facing the second user with the virtual button A pushed. Alternatively, the second user may see the first user's interface from a different perspective of the first user. For example, if the first user presses button A, then the second user can see that button A is pressed on the first user's copy of the user interface.

As mentioned earlier, the second user may also view navigation views or alternative perspective views of the image data set (e.g., breakout views). This might include seeing views of the image data set from perspectives that are different than where the second user is actually standing. These views might be a view at an angle to the second user's view, the views may be top and bottom views or other oblique views that may be useful to the medical procedure being undertaken. Accordingly, the second user may switch to or use a panel (e.g., a graphical panel or pane presented in the AR headset) having at least one alternative perspective view of the image data set as defined with respect the second user view and second user's position. Further, at least one navigational view may be presented to the second user. The navigational views may be at least one of: a view of the image data set that is orthogonal to the second user view, a custom perspective defined by the second user, a defined view of a medical guide or medical implement in the medical procedure, or a defined view that is aligned and locked to a body of a person (e.g., an axis of the body of the person). A navigation view may include thumbnail views on a user interface bar viewable through the first AR headset and/or second AR headset.

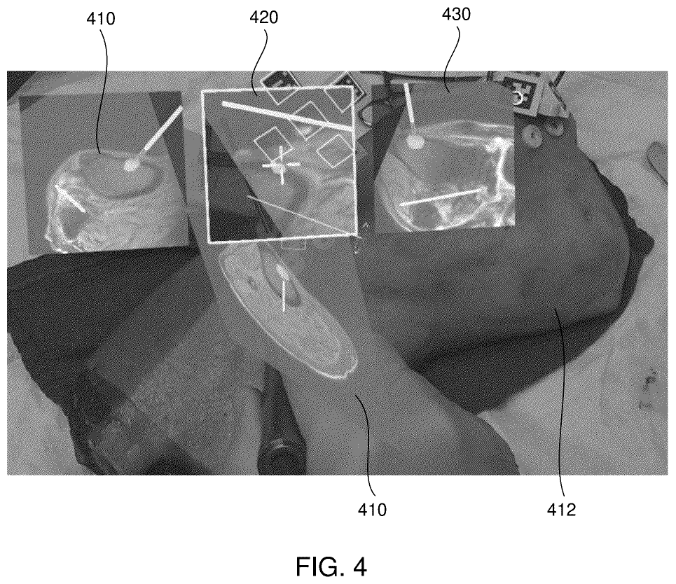

FIG. 4 illustrates an example of a slice 410 of a 3D image data set that may be displayed as an overlay to anatomy 412 using the AR headset. A heads up display of multiple breakout views 420, 430, 440 of the X-ray generated images is also displayed. The breakout view may show navigational views of a 3D slice of the 3D image data or the entire image data set desired by a medical professional (e.g., orthogonal to the AR headset view, etc.) as overlays. The breakout views can be navigational views illustrating 3D slices from other perspectives for viewing the overlaid 3D slice. For example, coronal, axial and sagittal views, etc. may be provided.

The navigation (or break-out) views can also be used by a medical professional to tell the medical professional how close they are to a target from a selected perspective. Otherwise, it can be difficult to determine a distance to and orientation of a medical instrument with respect to a target in three dimensions. In one configuration of this technology, the second medical professional may see the break-out view (e.g., in a mini view) from the same perspective(s) as the primary medical professional or first doctor. In another view configuration, the second medical professional can see the primary view and the break-out view(s) from the second medical professional's own perspective. In another alternative, the first and second medical professionals may want to see some of these elements aligned with or locked to the patient anatomy. Then both the first medical professional and second medical professional will see the primary view and/or breakout views the same way regardless of their physical location in the 3D coordinate system of an operating room. The opportunity to set the various views allows the medical professional to individually determine whether they want to see the views from their own perspective in the 3D coordinate system or from the view the primary doctor is currently seeing.

In another example, the second medical professional may select whether they want to see a graphical user interface from the first medical professional's perspective or from their own perspective. This may allow the second medical professional to see a menu or buttons that are their own menu, or the second AR headset of second medical professional can display the first medical professional's menu that is tilted toward the second medical professional. The view of the other medical professional's graphical user interface may also include all the individualized and/or personalized settings that are set for the other medical professional. For instance, allowing a second doctor to see the exact user interface for the first doctor is useful for training, recording, or documenting during the use of the AR headsets.

In another example configuration, the second doctor may see what the first doctor is seeing from a third perspective. The second doctor can set a point in space that is not the first doctor's perspective or the second doctor's perspective but is a third point in space where the doctor would like to view the 3D slices or image data set from.

In yet another configuration, the second medical professional can be contributing to what the first doctor is seeing. Accordingly, the second doctor may see the 3D slice and image data set as though the second doctor is in the place of the first doctor and is moving the needle. In this configuration, the second doctor may also see the first doctor's menus, navigation interface and other graphical user interface controls pointing toward the second doctor.

In a training situation, where two doctors both have AR headset then the second doctor may be trying assist and/or train the first doctor. If the second doctor is training the first doctor, then the second doctor may want to see the virtual overlays in the same way that the first doctor is seeing the virtual overlays. This means the second doctor may see a 3D slice, 3D image data, the first doctor's graphical user interface and/or other virtual objects from the first user's perspective. In addition, when the second doctor makes changes to the 3D slice or 3D image data in the first doctor's user interface, then those changes may be reflected in both the first doctor's and second doctor's views in their AR headsets. In contrast, if a second doctor is assisting a first doctor with a medical procedure but is not training the first doctor, then the second doctor may want to see the 3D slice, 3D image data and the user interface from the second doctor's own perspective.

In this technology, every virtual element that the doctors may interact with can be shared between the different headsets (e.g., two or more headsets). Some elements may be best displayed in one way, such as the 3D data set that is aligned with the anatomical structure of a patient. However, a slice or projection from a 3D data set can also be displayed at different coordinate locations in 3D coordinate system of the AR headset, as described earlier. Other elements in AR headset, such as navigation displays and user interface may be shown in an individualized way for each individual user or doctor based on the configurations described.

There may be at least three perspectives that can be provided to a medical professional:
1. In the first perspective case, the second doctor can see the virtual objects the first doctor is seeing from the first doctor's perspective (see the first doctor's interface and view of virtual overlays using the second doctor's AR headset);
2. In the second perspective case, the second doctor can set the configuration to seeing what the first doctor is doing from second doctor's perspective;
3. In a third perspective case, the second doctor may see their own UI and their own perspective for virtual objects and user interfaces overlaid on the scene.

In prior configurations of AR headsets, AR headsets that are in remote locations (e.g., are geographically separated) are able to share virtual objects that the users of both headsets can view and manipulate. However, in the situation where virtual objects are shared using geographically separate AR headsets, each user is viewing the virtual object from exactly the same perspective as other users. In contrast, the present technology allows users to select which type of virtual object they want to see and the perspective the user wants to see the virtual object from (e.g., another doctor or another user).

For an assisting surgeon there may be virtual items that the assisting surgeon wants to see customized from their own perspective, as opposed to trying to see things from the primary surgeon's (i.e., first doctor's) perspective. This technology is not describing remote medical assistance but rather the training of medical professionals (e.g., surgeons) and the ability for medical professionals to locally cooperate on surgical or medical procedures. This is because the medical professionals sharing the view can be in the same 3D coordinate space, as opposed to being in separate locations.

This technology is also different than the sharing of virtual objects that has previously existed in terms of data transfer. The sharing of interfaces between medical professionals in this technology is for training and assisting. There is no need for the AR headsets using the same 3D coordinate space to share bandwidth intensive imaging with each other because any virtual objects or virtual images (e.g., image data sets) do not need to be streamed between the AR headsets. Instead, the AR headsets can retrieve an image data set, virtual objects or virtual images from a central server or storage location to which the AR headsets are wirelessly and directly connected. Accordingly, the AR headsets of the present technology are not sharing virtual objects or virtual images across a computer network but the processes on the AR headsets can have shared control of: navigation of image data sets, moving of the image data sets, navigation and moving of slices of the image data sets, any other navigation for the image data sets and/or use of the graphical user interface controls. As a further example, the second medical professional's AR headset may send the first medical professional's AR headset: the parameters for a 3D slice that is being viewed, where the first medical professional's instrument should be, a depth for an instrument, a target object, a pointer or virtual instrument, etc. The AR headsets are not transferring data for virtual objects, but may share navigation data, perspective, and locational data of virtual objects. Immersively and cooperatively navigating through 3D image data from the first doctor's perspective or the second doctor's perspective is different than sharing virtual object data or image data sets between two AR headsets. Instead, this technology can share user interface controls, the coordinates of the controls, the coordinates of the AR headsets, the location of a slice, the slice being viewed, etc.

In the shared view technology, the AR headset can display what portions of an image data set or other virtual objects a first doctor is seeing to a second doctor. The navigation views and the oblique views can also show what the other doctor is seeing. However, the 3D image data set does not need to be streamed to the second doctor for each change in the navigation. The medical imaging data can be loaded independently of the positional or navigation data and prior to the navigation processes. This is because the location of first doctor can be sent to second doctor's headset and the perspective of the first doctor can be used to construct the immersive view for the second doctor.

The settings for the breakout or navigation view can also be sent to the second doctor and those views can be reconstructed or generated at the second AR headset of the second doctor. Thus, the data is being sent as a 3D construct to the AR headsets only one time, and then the AR headset can show the data using the defined perspectives. It is less desirable to try to stream large image data or detailed virtual objects between the AR headsets of individuals located in the same 3D coordinate system. Thus, the image data set can be sent once to each AR headset and then the image data set can be aligned to the body of the patient, but both the first doctor can see the image data set and the second doctor can see the image data set. Either doctor may control how the camera representing both doctors may be flying through or around the image data set, and either doctor can control the image data set. Either of doctors may control where the navigation views are and how the immersive view is presented, what the window level settings are, etc. This results in the streaming of a few kilobytes of positional and navigational data between the AR headsets but not megabytes or gigabytes of data for virtual objects or the 3D image data set. This is because sharing of the navigation instructions and positions of the AR headsets in the 3D coordinate system consumes much less data bandwidth than transferring the image data set of the patient. The amount of data sent between two headsets in the same 3D coordinate system may be reduced because the spatial positioning is shared between the two AR headsets and only smaller amounts of information need to shared, including the position of the second AR headset as compared to the first AR headset, using the X, Y, Z position of each AR headset.

In addition, the 3D controls for the breakout views or the navigation views may also be shared in the same ways as described above. For example, the second medical professional may see their own controls for the navigation views or breakout views. Alternatively, the second doctor can see and interact with the controls for the navigation views or breakout views (and the navigation views themselves) of the first doctor that is being trained or assisted.

In one example, the second doctor can see the 3D image data set from the first doctor's perspective or from the second doctor's own perspective in the 3D coordinate system. The senior surgeon could be training a junior surgeon. The junior surgeon can ask the AR headset to generate a sagittal slice and then create a virtual needle. Then the senior surgeon can show the junior surgeon how the drag the slice and manipulate both the physical needle tip and the virtual needle using the shared view and user interface controls. The senior surgeon may also change the slice thickness, and modify the window level. The junior surgeon may then be shown the target, for instance in the aorta, on their own headset and both the junior surgeon and senior surgeon can see the target marking. This may allow to senior surgeon to correct the views seen and change an incorrect needle insertion position or other incorrect surgical maneuvers. The senior surgeon may watch where the junior surgeon (e.g., resident) is placing the needle and what slice of the image data set is being viewed from their senior surgeon's perspective or the junior surgeon's perspective.

This technology may provide the ability to switch between views of the virtual objects in the 3D coordinate system. The senior surgeon may see one view and the junior surgeon may see another view. Then the senior surgeon may be able to switch between the junior surgeon's view of the virtual objects and/or user interface and their own perspective by toggling a user interface control or selecting a menu item through the AR headset. For instance, the senior surgeon may want to see the navigation views facing the junior surgeon, and then switch to see the navigation views facing the junior surgeon. In the case where there are three (3) or more surgeons in the room (e.g., up to N surgeons) the senior surgeon (or any surgeon with the appropriate permissions) may switch between doctor 1, doctor 2, doctor 3 and additional doctor views or viewing controls.

This technology may be applicable where the junior doctor is trying to navigate an instrument into a 3D object like a spine. If the senior doctor is on the opposing side of the patient, the senior physician (e.g., attending physician) can roughly see what the resident doing. However, what the senior physician really desires to see is if that junior doctor (e.g., resident) is on track to put the needle tip at the right location in the spine in three dimensions. This technology allows the senior doctor to see the same view of the image set data and virtual tool that the junior doctor (e.g., resident) is seeing, so the senior doctor can help adjust or alter the trajectory of the needle or other instrument which the junior doctor is controlling. This allows the senior doctor to determine whether the junior doctor is going into or through the right anatomy in the spine.

There are some 3D slice views of the image data set that may look different based on what the medical professional is looking at on the body of patient, particularly where these views are registered to the patient. For views of the image data set that are registered to the patient, these views may have a defined location and orientation in the 3D coordinate system. The example of an axial slice of the image data set that is aligned to the body of the patient will look the same from two opposite sides but flipped. However, an oblique view is typically set to dynamically face the medical professional or user as the medical professional moves around the body of the patient (i.e., the navigation views may be also be facing the user). In the present system, the second doctor may see the exact same view of an oblique view that the first doctor is seeing and the oblique view may not be aligned with the body of the patient for the second viewer. For example, this may mean an oblique view may not align with a patient's anatomical structures for the second doctor.

The second medical professional may also receive (or be sent) the instrument tracking location, regarding where the tip of a needle is in space as controlled by the first medical professional. Instructions can also be sent back and forth to control the view of the 3D or 2D data. For example, the second doctor may control the operation of a “fly through” for the image data set. Similarly, if the second doctor starts to scroll through slices of the image data set, then the AR headset can start the scrolling view for the first doctor or vice versa. It is not the images, 3D image data sets or virtual objects that are shared between the two AR headsets, it is the coordinate space of the instrument, the patient location, any annotations, any segmentation done, etc. Each medical professional may have the ability to reciprocally control and switch back and forth between the other medical professional's views of virtual objects and their user interface.

This technology provides a co-pilot feature that may be used between two or more doctors. By analogy, being a single airplane pilot is different using than a dual control plane. The present invention is a dual or multi-control system for an immersive 3D space. The two users or medical professionals (e.g., co-pilots) who are navigating the virtual overlays in the shared 3D space may be able to share: the 3D registration of the image data set, user interface controls, slicing, a 3D coordinate system, instrument locations and other information for the 3D image data set (e.g., a point cloud of data) they are navigating through.

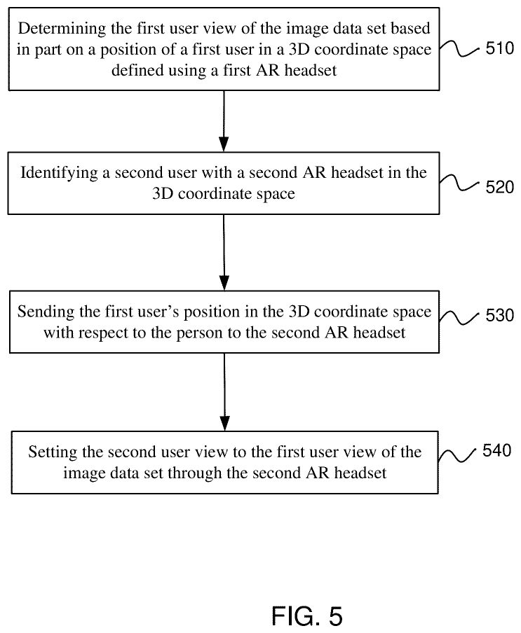

FIG. 5 is a flowchart illustrating a method for managing a first user view and a second user view of an image data set aligned with a body of a person (e.g., using at least one marker on a person or another alignment method) and AR headsets. One operation of the method may be determining the first user view of the image data set based in part on a position of a first user in a 3D coordinate space defined using a first AR headset, as in block 510.

A second user with a second AR headset in the 3D coordinate space may be identified, as in block 520. The first AR headset and the second AR headset may be using a common 3D coordinate system in a location or operating room. The system may be configured to let the second user and second AR headset share the virtual view, user interface controls, navigation controls or any other user interface controls and virtual aspects with the first user. The first user's position in the 3D coordinate space with respect to the person may be sent to the second AR headset, as in block 530.

The second user view can be set to the first user view of the image data set through the second AR headset, as in block 540. The second AR headset can use the 3D coordinate position of the first user to provide the view of the image data set to the second AR headset, where the view of the image data set displayed by the second AR headset may match the first headset. For example, at least a portion of the image data set can be displayed from a perspective that matches the first user view of the image data set. The portion of the image data set may be a slice of samples or voxels (e.g., at least one layer) from the image data set.

The second user may also be able to control the first user's interface for viewing and navigation of the image data set that is overlaid on a patient through the AR headset. This may include switching to a control interface of the first user to enable viewing and control of the image data set using the control interface of the first user. A representation of the first user view may be sent to the second AR headset of the second user. The representation may include instructions regarding at least one of: identifying at least one 3D slice viewed from a perspective of the first user, a location of the first user in the 3D coordinate space, a depth of at least one projection slice, a medical device location, instrument locations, virtual object locations, virtual instruments, or a pointer location.

The first user and the second user may be able share user interface functions from their own perspective. These shared user interface functions may be at least one of: altering a position of the image data set in the 3D coordinate system, moving to a different slice in the image data set, rotating a projection slice and using graphical user interface (GUI) controls from their own perspective. Similarly, the second user may perform functions from a perspective of the first user, including at least one of: altering a position of the image data set, dragging to a different slice in the image data set, changing the slice position, rotating a projection slice, and using graphical user interface (GUI) controls from the perspective of the second user.

In another configuration, the second user view may switch to a panel having at least one alternative perspective view (i.e., navigation views) of the image data set as defined by the first user view and/or first user's position. As a result, one or more additional navigational views of the first user are presented to the second user. The additional navigation views may include at least one of: a view of the image data set that is orthogonal to the first user view, a custom perspective set by the first user, a defined view of a medical guide, or a defined view that is locked to a body of a person. The navigation view may include thumbnail views on a user interface bar viewable through the first AR headset or second AR headset.

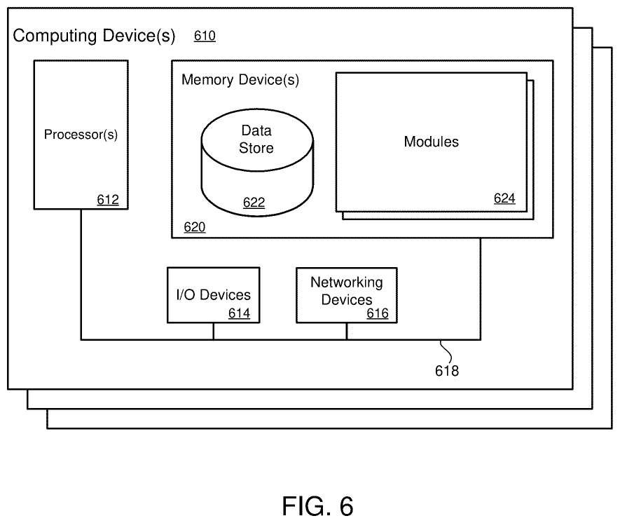

FIG. 6 illustrates a computing device 610 on which modules of this technology may execute. The computing device 610 is illustrated on which a high level example of the technology may be executed. The computing device 610 may include one or more processors 612 that are in communication with memory devices 620. The computing device may include a local communication interface 618 for the components in the computing device. For example, the local communication interface may be a local data bus and/or any related address or control busses as may be desired.

The memory device 620 may contain modules 624 that are executable by the processor(s) 612 and data for the modules 624. The modules 624 may execute the functions described earlier. A data store 1022 may also be located in the memory device 620 for storing data related to the modules 624 and other applications along with an operating system that is executable by the processor(s) 612.

Other applications may also be stored in the memory device 620 and may be executable by the processor(s) 612. Components or modules discussed in this description that may be implemented in the form of software using high programming level languages that are compiled, interpreted or executed using a hybrid of the methods.

The computing device may also have access to I/O (input/output) devices 614 that are usable by the computing devices. An example of an I/O device is a display screen that is available to display output from the computing devices. Other known I/O device may be used with the computing device as desired. Networking devices 616 and similar communication devices may be included in the computing device. The networking devices 1016 may be wired or wireless networking devices that connect to the internet, a LAN, WAN, or other computing network.

The components or modules that are shown as being stored in the memory device 620 may be executed by the processor 612. The term “executable” may mean a program file that is in a form that may be executed by a processor 612. For example, a program in a higher level language may be compiled into machine code in a format that may be loaded into a random access portion of the memory device 620 and executed by the processor 612, or source code may be loaded by another executable program and interpreted to generate instructions in a random access portion of the memory to be executed by a processor. The executable program may be stored in any portion or component of the memory device 620. For example, the memory device 620 may be random access memory (RAM), read only memory (ROM), flash memory, a solid state drive, memory card, a hard drive, optical disk, floppy disk, magnetic tape, or any other memory components.

The processor 612 may represent multiple processors and the memory 620 may represent multiple memory units that operate in parallel to the processing circuits. This may provide parallel processing channels for the processes and data in the system. The local interface 618 may be used as a network to facilitate communication between any of the multiple processors and multiple memories. The local interface 618 may use additional systems designed for coordinating communication such as load balancing, bulk data transfer, and similar systems.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions, and may even be distributed over several different code segments, among different programs, and across several memory devices. Similarly, operational data may be identified and illustrated herein within modules, and may be embodied in any suitable form and organized within any suitable type of data structure. The operational data may be collected as a single data set, or may be distributed over different locations including over different storage devices. The modules may be passive or active, including agents operable to perform desired functions.

The technology described here can also be stored on a computer readable storage medium that includes volatile and non-volatile, removable and non-removable media implemented with any technology for the storage of information such as computer readable instructions, data structures, program modules, or other data. Computer readable storage media include, but is not limited to, RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which can be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared, and other wireless media. The term computer readable media as used herein includes communication media.

Furthermore, the described features, structures, or characteristics may be combined in any suitable manner in one or more examples. In the preceding description, numerous specific details were provided, such as examples of various configurations to provide a thorough understanding of examples of the described technology. One skilled in the relevant art will recognize, however, that the technology can be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements can be devised without departing from the spirit and scope of the described technology.

What is claimed is:

1. A method for managing a first user view and a second user view of an image data set aligned with a body of a person using AR headsets, comprising:
determining the first user view of the image data set based in part on a position of a first user in a 3D coordinate space defined using a first AR headset;
identifying a second user with a second AR headset in the 3D coordinate space;
sending a first user's position in the 3D coordinate space with respect to the person to the second AR headset; and
setting the second user view to the first user view of the image data set through the second AR headset.

2. The method as in claim 1, further comprising displaying at least a portion of the image data set from a perspective that matches the first user view of the image data set.

3. The method as in claim 1, further comprising allowing a first user to control the second user view of the image data set or navigation.

4. The method as in claim 1, further comprising allowing a second user to control the first user view of the image data set.

5. The method as in claim 1, wherein data representing the first user view is sent to the second AR headset of the second user including at least one of:
at least one projection slice viewed from a perspective of the first user,
a location of the first user in the 3D coordinate space,
a depth of at least one projection slice,
a medical device location,
object locations; or
a pointer location.

6. The method as in claim 1, wherein the first user and the second user share user interface functions from their own perspective that are at least one of:
altering a position of the image data set in the 3D coordinate system,
moving to a different slice in the image data set,
rotating a projection slice, and
using graphical user interface (GUI) controls from their own perspective.

7. The method as in claim 1, wherein the second user performs functions from a perspective of the first user that are at least one of:
altering a position of the image data set,
dragging to a different slice in the image data set,
rotating a projection slice, and
using graphical user interface (GUI) controls from the perspective of the second user.

8. The method as in claim 1, wherein the first AR headset and the second AR headset are using a common 3D coordinate system in a location.

9. The method as in claim 1, further comprising switching to a panel having at least one alternative perspective view of the image data set as defined by the second user view and second user's position.

10. The method as in claim 1, wherein at least one navigational view is presented to the second user.

11. The method as in claim 10, wherein the at least one navigation view may be at least one of: a view of the image data set that is orthogonal to the second user view, a custom perspective set by the second user, a defined view of a medical guide, or a defined view that is locked to a body of a person.

12. The method as in claim 10, wherein the at least one navigation view includes thumbnail views on a user interface bar viewable through the first AR headset or the second AR headset.

13. The method as in claim 1, wherein the at least a portion of the image data set is a slice from the image data set.

14. A system for managing a first user view and a second user view of an image data set aligned with a body of a person using AR headsets, comprising:
at least one processor;
at least one memory device including a data store to store a plurality of data and instructions that, when executed, cause the system and processor to:
determine the first user view of the image data set by a first user based in part on a first user's position in a 3D coordinate space defined using a first AR headset;
identify a second user with a second AR headset in the 3D coordinate space;
send the first user's position in the 3D coordinate space with respect to the person and image data set to the second AR headset;
set the second user view to the first user view of the image data set through the second AR headset; and
display at least a portion of the image data set from a perspective that matches the first user view of the image data set.

15. The system as in claim 14, further comprising switching to a control interface of a first user to enable viewing and control of the image data set presentation using the control interface of the first user.

16. The system as in claim 14, wherein data representing the first user view is sent to the second AR headset of the second user including at least one of:
at least one projection slice viewed from a first user's perspective,
a location of the first user in the 3D coordinate system,
a depth of at least one projection slice,
object locations, or
a pointer location.

17. The system as in claim 14, wherein the first user and the second user share user interface functions from their own perspectives that are at least one of:
altering a position of the image data set in the 3D coordinate system,
moving to a different slice in the image data set,
rotating a projection slice, and
using graphical user interface (GUI) controls from their own perspective.

18. The system as in claim 14, wherein the second user performs functions from a perspective of the first user that are at least one of:
altering a position of the image data set,
dragging to a different slice in the image data set,
rotating a projection slice, and
using graphical user interface (GUI) controls from a first user's perspective.

19. The system as in claim 14, wherein the first AR headset and the second AR headset are using a common 3D coordinate system in a location.

20. The system as in claim 14, further comprising switching to a panel having at least one alternative perspective view of the image data set as defined by the second user view and second user's position.

21. The system as in claim 14, wherein at least one navigational view is presented to the second user.

22. The system as in claim 21, wherein the at least one navigation view may be at least one of: a view of the image data set that is orthogonal to the second user view, a custom perspective set by the second user, a defined view of a medical guide, or a defined view that is locked to a body of a person.

23. The system as in claim 21, wherein the at least one navigation view includes thumbnail views on a user interface bar viewable through the first AR headset.

24. A machine-readable storage medium having instructions embodied thereon, the instructions when executed by one or more processors, cause the one or more processors to perform a process comprising:
determining a first user view of an image data set based in part on a position of a first user in a 3D coordinate space defined using a first AR headset;
identifying a second user with a second AR headset in the 3D coordinate space;
sending a first user's position in the 3D coordinate space with respect to a person to the second AR headset; and
setting a second user view to the first user view of the image data set through the second AR headset.

25. The machine-readable storage medium as in claim 24, further comprising displaying at least a portion of the image data set from a perspective that matches the first user view of the image data set.

26. The machine-readable storage medium as in claim 24, further comprising switching to a control interface of the first user to enable viewing and control of the image data set using the control interface of the first user.

27. The machine-readable storage medium as in claim 24, wherein data representing the first user view is sent to the second AR headset of the second user including at least one of:
at least one projection slice viewed from a perspective of the first user,
a location of the first user in the 3D coordinate space,
a depth of at least one projection slice,
a medical device location,
object locations; or
a pointer location.

28. The machine-readable storage medium as in claim 24, wherein the first user and the second user share user interface functions from their own perspective that are at least one of:
altering a position of the image data set in the 3D coordinate system,
moving to a different slice in the image data set,
rotating a projection slice, and
using graphical user interface (GUI) controls from their own perspective.

29. The machine-readable storage medium as in claim 24, wherein the second user performs functions from a perspective of the first user that are at least one of:
altering a position of the image data set,
dragging to a different slice in the image data set,
rotating a projection slice, and
using graphical user interface (GUI) controls from the perspective of the second user.

30. The machine-readable storage medium as in claim 24, wherein the first AR headset and the second AR headset are using a common 3D coordinate system in a location.

31. The machine-readable storage medium as in claim 24, further comprising switching to a panel having at least one alternative perspective view of the image data set as defined by the second user view and second user's position.

32. The machine-readable storage medium as in claim 24, wherein at least one navigational view is presented to the second user.

33. The machine-readable storage medium as in claim 32, wherein the at least one navigation view may be at least one of: a view of the image data set that is orthogonal to the second user view, a custom perspective set by the second user, a defined view of a medical guide, or a defined view that is locked to a body of a person.

34. The machine-readable storage medium as in claim 32, wherein the at least one navigation view includes thumbnail views on a user interface bar viewable through the first AR headset or the second AR headset.

35. The machine-readable storage medium as in claim 24, wherein the at least a portion of the image data set is a slice from the image data set.
